(19) United States
(12) Plant Patent Application Publication
(10) Pub. No.: US 2003/0028935 P1
(43) Pub. Date: Feb. 6, 2003

(54) Raspberry plant named 'Esta'

(76) Inventors: Harry J. Swartz, Laurel, MD (US); Joseph A. Fiola, Keedysville, MD (US); Herbert D. Stiles, Blackstone, VA (US); Brian R. Smith, River Falls, WI (US)

Correspondence Address: JAMES A. POULOS, III, UNIVERSITY OF MARYLAND, OFFICE OF TECHNOLOGY COMMERCIALIZATION, 6200 BALTIMORE AVENUE, SUITE 300, RIVERDALE, MD, 20737-1054, US

(21) Appl. No.: 09/917,240

(22) Filed: Jul. 30, 2001

(51) Int. Cl. A01H 5/00

(52) U.S. Cl. PLT/204

(57) ABSTRACT

The present invention is a new and distinct spring bearing red raspberry cultivar named ‘Esta’ (also tested as GEL-114), which is capable of producing large numbers of fruit in the early spring season, the fruit being similar to that of the standard cultivars. The cultivar is characterized by moderate suckering ability, sparse red thorns and very vigorous canes and leaves which are commonly free of fungal diseases until leaves senesce in the Fall. ‘Esta’ has resistance to fluctuating winter and spring temperatures, this has resulted in excellent winter survivability in the Mid-Atlantic States. Because of the rapid growth of primocanes, fruit is usually shaded during harvest and fruit rot can occur if the primocane canopy is not separated from the fruiting canopy.

### FIELD OF THE INVENTION

This invention concerns a new and distinct cultivar of raspberry plant with a botanical name of Rubus ideaus L.

### DESCRIPTION OF RELATED PRIOR ART

Several cultivars of raspberry plant are known. For instance, raspberry cultivars named ‘Anne’, ‘Caroline’ and ‘Lauren’ have been described in U.S. Plant Pat. Nos. 10,411, 10,412 and 10,610, respectively. The new and distinct cultivar of the present invention is a raspberry plant named ‘Esta’. This new and distinct cultivar of the present invention differs from ‘Anne’ in bearing red fruit, rather than golden fruit. ‘Esta’ can be distinguished from ‘Caroline’ in that ‘Esta’ is mostly spring bearing, but ‘Caroline’ is fall bearing. ‘Caroline’ canes are also less vigorous and more susceptible to a number of cane and leaf fungi. ‘Esta’ and ‘Lauren’ are both spring bearing red raspberry cultivars, but ‘Esta’ can be distinguished from ‘Lauren’ because ‘Esta’ fruit are smaller, leaves and canes are less susceptible to fungal diseases and ‘Esta’ canes are less susceptible to fluctuating temperatures in the late winter and early spring.

### ORIGIN OF THE NEW CULTIVAR

The new cultivar of spring bearing red raspberry originated from a controlled cross at the Scottish Crop Research Institute in Dundee Scotland, United Kingdom, by Harry Jan Swartz. The cross “EL” was ‘SCRI 8616B6’ (unpatented)×‘Southland’ (unpatented) and was made in the summer of 1986. This year was designated “G” as part of the University of Maryland at College Park; Rutgers University of New Brunswick, N.J.; Virginia Polytechnic Institute and State University, Southern Piedmont Agricultural Research and Education Center at Blackstone; and the University of Wisconsin at River Falls Cooperative Bramble Breeding Program. The clone was first selected in 1989 at the Western Maryland Research and Education Center of the University of Maryland located at Keedysville, Md. and was therefore designated “-114”. Thus, the complete breeding designation was “GEL-114”.

### SUMMARY OF THE NEW CULTIVAR

This application relates to a new and distinct red fruited, spring bearing raspberry cultivar, botanically known as Rubus ideaus L. The following characteristics are outstanding:

1. The plant is very productive in the Mid-Atlantic States due to its resistance to fluctuating temperatures in the late winter and early spring.

2. Canes are very vigorous and resistant to most cane and leaf attacking fungi. Cane growth is typically 6-10 feet.

3. Fruit is early to ripen in the spring, ripening with several early cultivars, for example ‘Lauren’ (PP10,610) and ‘Reveille’ (unpatented).

The following characteristics are useful in distinguishing this cultivar and can be useful for cultivar identification.

1. Plants are moderately suckering and very vigorous, growing up to 10 feet or taller when mature.

2. Canes are very resistant to cane diseases and leaves are somewhat darker than other cultivars.

3. The fruit is produced in the early season from the floricanes. Primocane produced fruit is common if the cultivar is grown in warmer climates or where the growing season is long (>200 days).

4. Fruit is moderate sized with good cohesion and firmness and a strong flavor typical of European cultivars.

### BRIEF DESCRIPTION OF THE PHOTOGRAPHS

The accompanying photographs show typical characteristics of the new variety:

FIGS. 1a and 1b show respectively ‘Esta’ and ‘Canby’ (unpatented) leaves with leaf fungi. These pictures were taken in October, 1999 at, South Deerfield, Mass.

FIG. 2 shows the type and density of thorns on an ‘Esta’ primocane.

FIG. 3 shows a close-up coloring and size of ‘Esta’ leaves, the ruler is 6 inches long.

FIG. 4 shows a fruiting cluster of ‘Esta’, showing the exposure of the fruit.

FIG. 5 shows development of ‘Esta’ flowers and fruit.

FIGS. 6a and 6b show the color, size, uniformity, shape and narrowness of cavity of harvested ‘Esta’ fruit.

FIG. 7 shows an ‘Esta’ plant in fruit in June 1999, the canopy top is at 6′ feet height, the plants are being grown on a floricane separated trellis system and are in their third growing season in Colt's Neck, N.J.

### DESCRIPTION OF THE NEW CULTIVAR

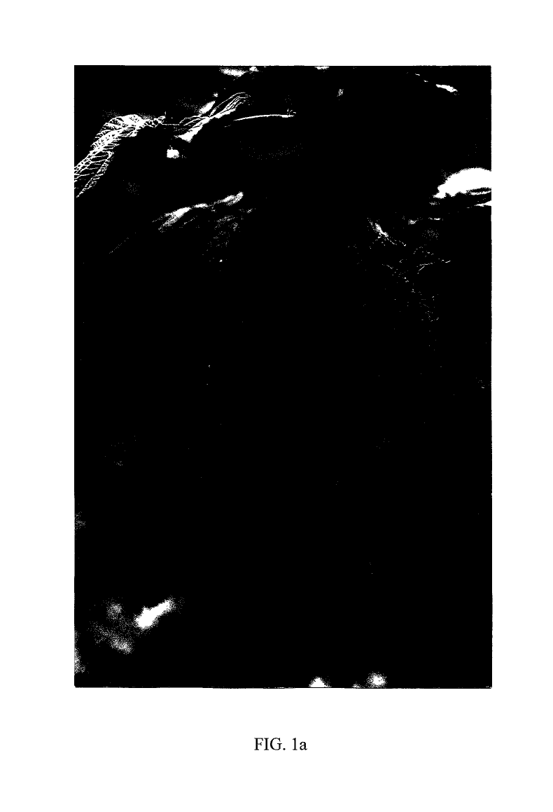

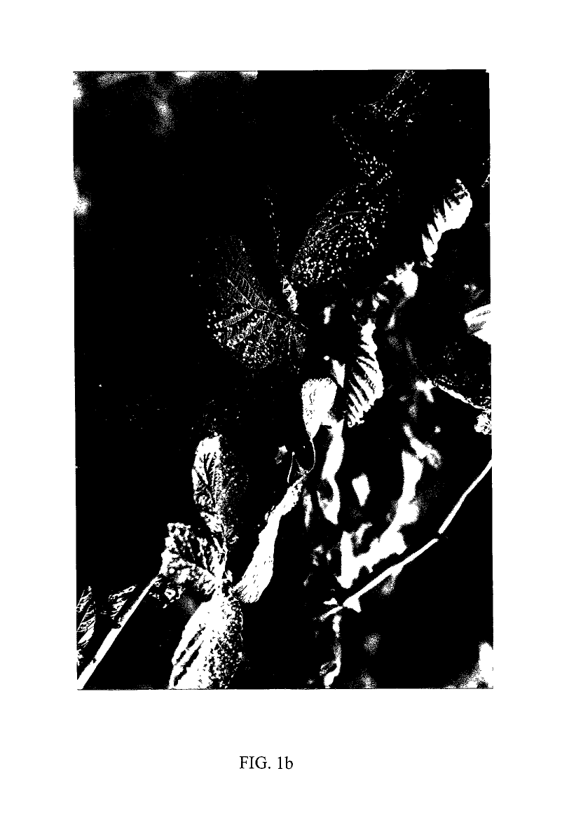

The following is a detailed description of the new cultivar, including fruit production, together with the cultivar's morphological characteristics. The characteristics of the cultivar were compared other standards used in the Mid-Atlantic Region of the U.S. The description is based on information provided by cooperating scientists and growers from plants grown in fields at Cream Ridge and Colt's Neck, N.J., South Deerfield, Mass. and Millersville, Pa., and from plants grown in the greenhouses at College Park, Md. ‘Esta’ produces a moderate number of root- and crown- suckers, 33 per 10 ft of row in Colt's Neck, N.J.; but higher numbers are common in other listed locations. These numbers are commonly similar to ‘Lauren’ (U.S. Plant Pat. No. 10,610), ‘Tulameen’ (unpatented) and “Sentry’ (unpatented), but greater than ‘Titan’ (U.S. Plant Pat. No. 5,404), ‘Claudia’ (patent applied for) and ‘AmosH’ (unpatented), typical cultivars tested or grown in the eastern United States. Unlike any other cultivar known to us, ‘Esta’ primocanes are very resistant to several leaf and cane infesting fungi, such as powdery mildew, anthracnose, cane botrytis, spur and cane blight and late leaf rust (see FIGS. 1a and 1b). The plant's reaction to Phytophthora fragarae root rot is moderately susceptible, based on field reaction, not in controlled testing.

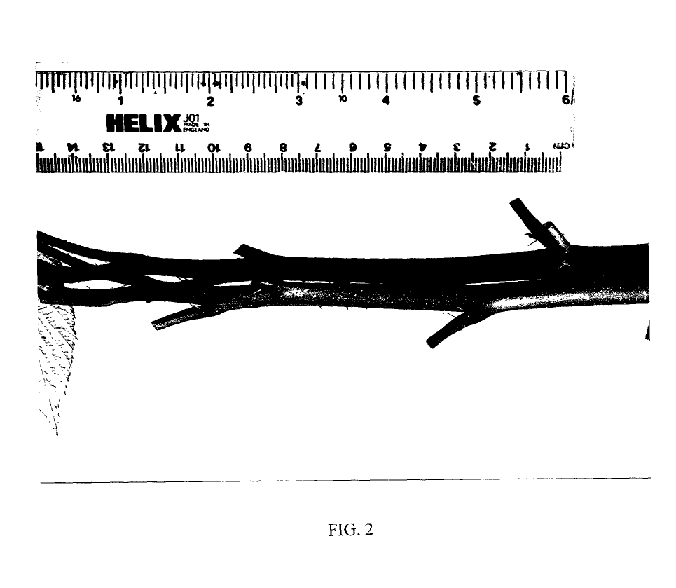

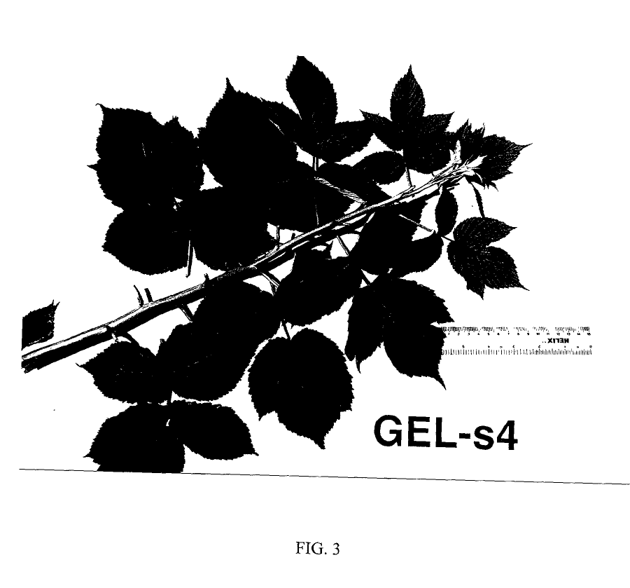

During the growing season, primocanes and flower truss axes from floricanes are yellow green colored (Royal Horticultural Society plate 145A) with a purple red blush (Royal Horticultural Society plate 59A) in full sun (see FIGS. 2, 3 and 4). Primocanes are usually unbranched, erect to arching and very vigorous. Floricanes are not exfoliating, except for an occasional long narrow fissure at their base. Floricane color is dark brown and consistent along the entire length of the cane, resembling Royal Horticultural Society plate 175A. Thorns are very sparse in density, 1 mm to 2 mm in length and red (Royal Horticultural Society plate 59B) from their base to their apex (see FIG. 2). Leaf upper surfaces are dark green, most closely in hue to Royal Horticultural Society Color Plate 137A, while the lower surface of the leaf is pubescent giving this surface a greyer color (Royal Horticultural Society plate 1 94B). Leaves are trifoliolate to pentafoliolate and average 10 cm from the distal end of the petiole to the distal end of the terminal leaflet (see FIG. 3). The basal leaflets average 13 cm. from terminal point to point. Petiole length is typically 4 cm. Leaf serration is common for most cultivars of red raspberry and cannot be used to distinguish this cultivar.

Floricanes suffer cold injury in mid winter if temperatures fall below −20° F. Floricanes are resistant to fluctuating temperatures, for example, if a warm spring is interrupted by an unusual period of freezing temperatures. Canes can flower from all live buds in April to May depending on latitude, and fruit from mid June to late July in the eastern U.S. This ripeness period is 1-2 days earlier than ‘Lauren’ (U.S. Plant Pat. No. 10,610) and ‘Reveille’ (unpatented), and 1-2 weeks earlier than ‘Titan’ (U.S. Plant Pat. No. 5,404) and ‘Tulameen’ (unpatented).

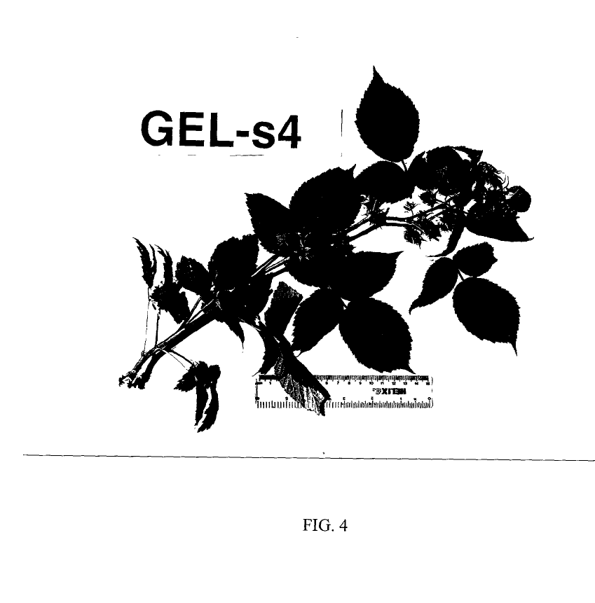

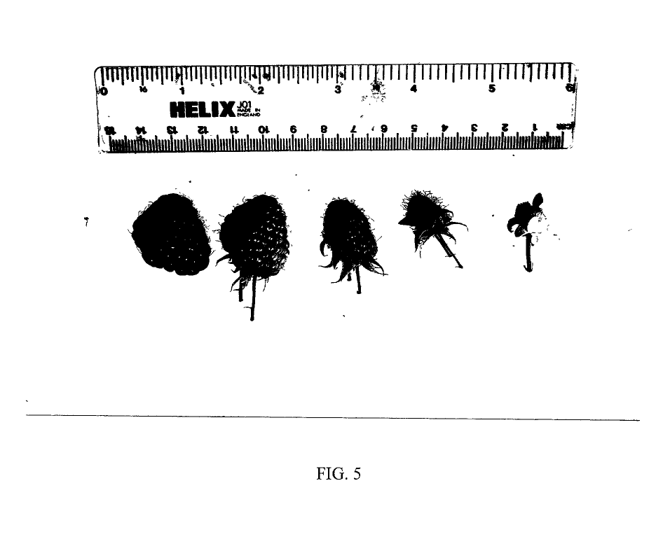

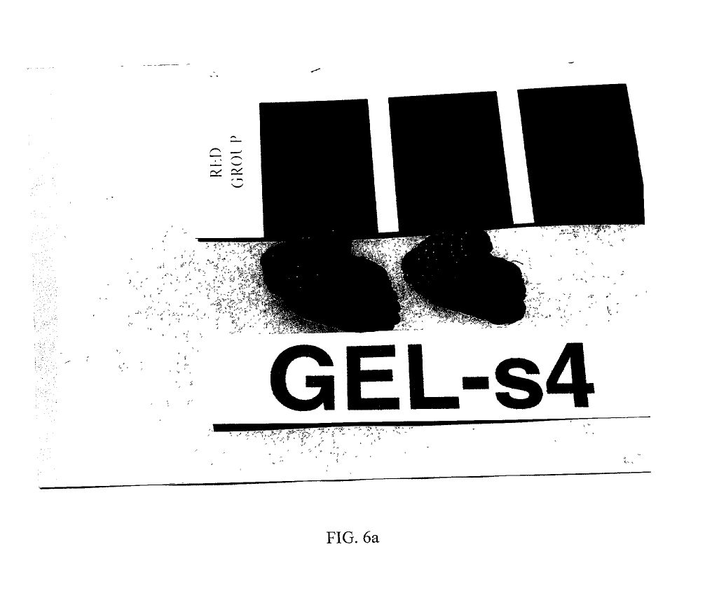

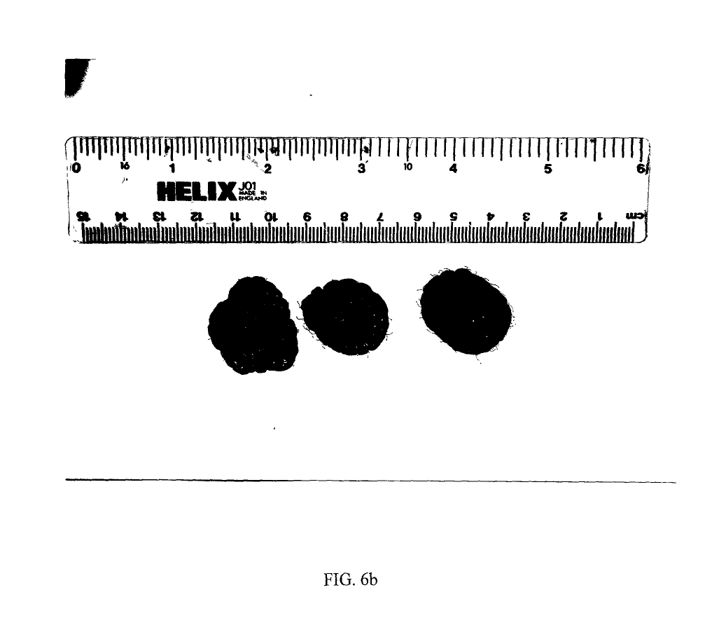

Flower morphology and early fruit morphology is typical of most red raspberry cultivars (unscented, 5 white petals persisting less than 5 days post pollination, 0.6 cm long and resembling Royal Horticultural Society plate 155D; 5 green sepals resembling Royal Horticultural Society plate 194B and 1.1 cm long) and cannot be used to identify ‘Esta’ (see FIG. 4 and 5). Fruit trusses have typical cymose clusters with 6 to over 20 fruit well spaced on a truss axis (see FIG. 4). Twenty-five days after pollination, fruit is distinguishable for this variety. External and internal color of the fruit is moderately red when ripe, closely resembling Royal Horticultural Society color plate number 46A (see FIGS. 6a and 6b). Fruit has a very slight pubescence, producing a slightly glossy appearance. Fruit is round conic, moderate sized (see Table 1) and symmetrical (see FIGS. 6a and 6b). Drupelets are held together tightly. The collar is very uniform, commonly lacking a large notch. The cavity width is similar to large-fruited cultivars (see FIG. 6). The fruit readily separates from the plant's recepticle . The fruit does not break down after at least one week in common storage at 40° F.; however, if hot temperatures (>90° F.) occur at harvest, fruit firmness is reduced. The fruit is not resistant to Botrytis induced rot. Flavor is strong and sweet and characteristic of red or purple red raspberries, especially cultivars released in Europe. Fall fruit, that is, fruit borne on the top of the primocanes, occurs in this cultivar when the growing season is >200 days. Fruit size and characteristics are similar to that of summer grown fruit.

### FRUIT PRODUCTION

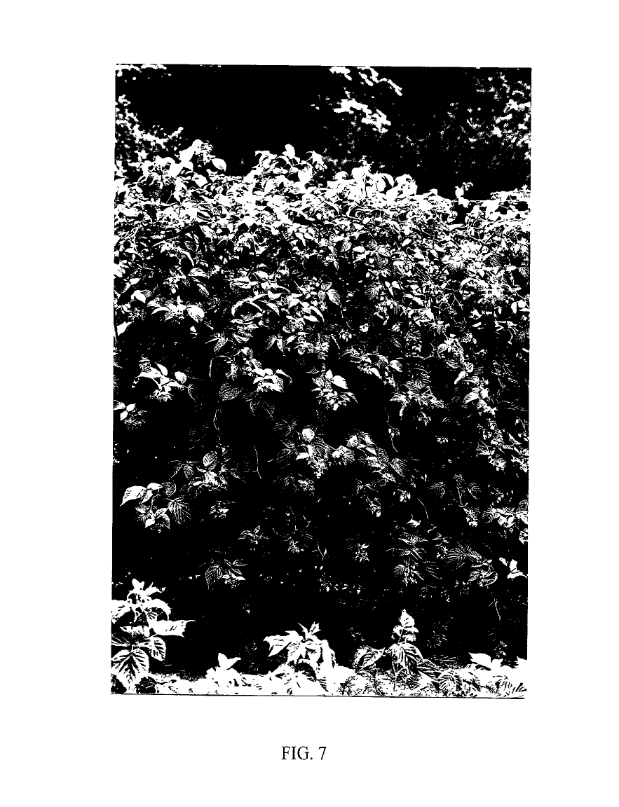

‘Esta’ has been tested in a grower trial in Millersville, Pa. The following data were collected in the Summer from 1997, the first year of production, to 1999. Mid winter temperatures were below 0° F. in the winter of 1997-1998, resulting in complete cane death in ‘Tulameen’, therefore, no data (N.D.) was obtained for fruit size in 1998. The size of ‘Esta’ primocanes, and its average floricane truss productivity should result in higher than normal yield. However, the ‘Esta’ primocane canopy is often very dense and should be separated from the floricane canopy to enhance aeration and reduce fungal contamination of fruit. Recommended training and trellis construction includes the Gjerde and Stiles Shift Trellis. Separated ‘Esta’ floricane canopies can result in highly productive plantings (see FIG. 7).

TABLE 1
Comparison of fresh fruit characteristics of ‘Esta’

| | Yield in lbs/acre | | | | Fruit Weight (grams) |
|---|---|---|---|---|---|
| | 1997 | 1998 | 1999 | average | 1998 |
| Esta | 3533 | 6014 | 3432 | 4326 | 2.6 |
| Lauren | 4549 | 8947 | N.D. | 6748 | 3.3 |
| Claudia | N.D. | 8650 | 5465 | 7057 | 3.8 |
| Amos H | 5905 | 7738 | 10475 | 8039 | 3.0 |
| Tulameen | 1743 | 0 | 7273 | 3005 | N.D. |

‘Esta’ has been produced by tissue culture and field suckering since 1991 at, South Deerfield, MA and the Department of Natural Resource Sciences, University of Maryland, College Park, MD. Over that period, no off-type of ‘Esta’ has been observed or reported to us. Tissue culture explants were obtained from lateral bud meristems and multiplication medium contained 3 to 15 micromoles benzyl adenine.

What is claimed is:

1. A new and distinct spring bearing red raspberry plant known as ‘Esta’ as described herein, illustrated and identified by the characteristics set forth above.
